SEQUENCE LISTING (60 sequences)

SEQ ID NO: 1 - DNA, 18 bp, Artificial Sequence
  RNA34 forward primer sequence
agccccctcactcccaaa

SEQ ID NO: 2 - DNA, 21 bp, Artificial Sequence
  RNA34 reverse primer sequence
gggtgctgtgcttctgtgaac

SEQ ID NO: 3 - DNA, 25 bp, Artificial Sequence
  p21 forward primer sequence
ttctgctgtctctcctcagatttct

SEQ ID NO: 4 - DNA, 21 bp, Artificial Sequence
  p21 reverse primer sequence
ggattagggcttcctcttgga

SEQ ID NO: 5 - DNA, 22 bp, Artificial Sequence
  p21 TaqMan probe sequence
ccactccaaacgccggctgatc

SEQ ID NO: 6 - DNA, 22 bp, Artificial Sequence
  GADD153 forward primer sequence
agaaccaggaaacggaaacaga

SEQ ID NO: 7 - DNA, 20 bp, Artificial Sequence
  GADD 153 reverse primer sequence
tctccttcatgcgctgcttt

SEQ ID NO: 8 - DNA, 18 bp, Artificial Sequence
  Apaf-1 forward primer sequence
tgcgctgctctgccttct

SEQ ID NO: 9 - DNA, 21 bp, Artificial Sequence
  Apaf-1 reverse primer sequence
ccatgggtagcagctccttct

SEQ ID NO: 10 - DNA, 25 bp, Artificial Sequence
  SUMO-1 forward primer sequence
gggtcagagaattgctgataatcat

SEQ ID NO: 11 - DNA, 22 bp, Artificial Sequence
  SUMO-1 reverse primer sequence
ccccgtttgttcctgataaact

SEQ ID NO: 12 - DNA, 23 bp, Artificial Sequence
  Bfl-1 forward primer sequence
cacaggagaatggataaggcaaa

SEQ ID NO: 13 - DNA, 23 bp, Artificial Sequence
  Bfl-1 reverse primer sequence
catccagccagatttaggttcaa

SEQ ID NO: 14 - DNA, 17 bp, Artificial Sequence
  BCL-W forward primer sequence
tccggcgcaccttctct

SEQ ID NO: 15 - DNA, 23 bp, Artificial Sequence
  BCL-W reverse primer sequence
cccaaagacaaagaaggctacaa

SEQ ID NO: 16 - DNA, 21 bp, Artificial Sequence
  Bcl-2 forward primer sequence
catgtgtgtggagagcgtcaa

SEQ ID NO: 17 - DNA, 21 bp, Artificial Sequence
  Bcl-2 reverse primer sequence
gccggttcaggtactcagtca

SEQ ID NO: 18 - DNA, 20 bp, Artificial Sequence
  PUMA forward primer sequence
gggcccagactgtgaatcct

SEQ ID NO: 19 - DNA, 24 bp, Artificial Sequence
  PUMA reverse primer sequence
acgtgctctctctaaacctatgca

SEQ ID NO: 20 - DNA, 19 bp, Artificial Sequence
  TaqMan probe sequence
ccccgccccatcaatccca

SEQ ID NO: 21 - DNA, 20 bp, Artificial Sequence
  NOXA forward primer sequence
ctcaggaggtgcacgtttca

SEQ ID NO: 22 - DNA, 18 bp, Artificial Sequence
  NOXA reverse primer sequence
ttccaagggcacccatga

SEQ ID NO: 23 - DNA, 19 bp, Artificial Sequence
  HRK forward primer sequence
gggagcccagagcttgaaa

SEQ ID NO: 24 - DNA, 23 bp, Artificial Sequence
  HRK reverse primer sequence
gcgctgtctttactctccacttc

SEQ ID NO: 25 - DNA, 23 bp, Artificial Sequence
  BIM forward primer sequence
tccaggacagttggatattgtca

SEQ ID NO: 26 - DNA, 23 bp, Artificial Sequence
  BIM reverse primer sequence
taaggagcaggcacagagaaaga

SEQ ID NO: 27 - DNA, 22 bp, Artificial Sequence
  BNIP3 forward primer sequence
aaatattccccccaaggagttc

SEQ ID NO: 28 - DNA, 19 bp, Artificial Sequence
  BNIP3 reverse primer sequence
cgctcgtgttcctcatgct

SEQ ID NO: 29 - DNA, 22 bp, Artificial Sequence
  BIK forward primer sequence
tcctatggctctgcaattgtca

SEQ ID NO: 30 - DNA, 21 bp, Artificial Sequence
  BIK reverse primer sequence
ggcaggagtgaatggctcttc

SEQ ID NO: 31 - DNA, 28 bp, Artificial Sequence
  BID forward primer sequence
catacactttttctctttccatgacatc

SEQ ID NO: 32 - DNA, 20 bp, Artificial Sequence
  BID reverse primer sequence
gggcatcgcagtagcttctg

SEQ ID NO: 33 - DNA, 22 bp, Artificial Sequence
  BAD forward primer sequence
caggcctatgcaaaaagaggat

SEQ ID NO: 34 - DNA, 18 bp, Artificial Sequence
  BAD reverse primer sequence
cgcaccggaagggaatct

SEQ ID NO: 35 - DNA, 18 bp, Artificial Sequence
  Bcl-Xs forward primer sequence
ggcaggcgacgagtttga

SEQ ID NO: 36 - DNA, 27 bp, Artificial Sequence
  Bcl-Xs reverse primer sequence
gttcccatagagttccacaaaagtatc

SEQ ID NO: 37 - DNA, 22 bp, Artificial Sequence
  BOK forward primer sequence
tcacatgctggttgcttaatcc

SEQ ID NO: 38 - DNA, 19 bp, Artificial Sequence
  BOK reverse primer sequence
gcacaaggaccccatcaca

SEQ ID NO: 39 - DNA, 21 bp, Artificial Sequence
  BAK forward primer sequence
cacggcagagaatgcctatga

SEQ ID NO: 40 - DNA, 20 bp, Artificial Sequence
  BAK reverse primer sequence
cccaattgatgccactctca

SEQ ID NO: 41 - DNA, 22 bp, Artificial Sequence
  BAX forward primer sequence
tttctgacggcaacttcaactg

SEQ ID NO: 42 - DNA, 18 bp, Artificial Sequence
  BAX reverse primer sequence
ggtgcacagggccttgag

SEQ ID NO: 43 - DNA, 19 bp, Artificial Sequence
  LRP forward primer sequence
cagctggccatcgagatca

SEQ ID NO: 44 - DNA, 21 bp, Artificial Sequence
  LRP reverse primer sequence
tccagtctctgagcctcatgc

SEQ ID NO: 45 - DNA, 19 bp, Artificial Sequence
  MRP forward primer sequence
caatgctgtgatggcgatg

SEQ ID NO: 46 - DNA, 23 bp, Artificial Sequence
  MRP reverse primer sequence
gatccgattgtctttgctcttca

SEQ ID NO: 47 - DNA, 19 bp, Artificial Sequence
  Beta-actin forward primer sequence
ttcaacaccccagccatgt

SEQ ID NO: 48 - DNA, 22 bp, Artificial Sequence
  Beta-actin reverse primer sequence
gtggtacgaccagaggcataca

SEQ ID NO: 49 - DNA, 22 bp, Artificial Sequence
  Beta-actin forward primer sequence
tctgtgtggattggtggctcta

SEQ ID NO: 50 - DNA, 21 bp, Artificial Sequence
  Beta-actin reverse primer sequence
ctgcttgctgatccacatctg

SEQ ID NO: 51 - DNA, 19 bp, Artificial Sequence
  Beta-actin forward primer sequence
ccctggctcctagcaccat

SEQ ID NO: 52 - DNA, 20 bp, Artificial Sequence
  Beta-actin reverse primer sequence
gagccaccaatccacacaga

SEQ ID NO: 53 - DNA, 23 bp, Artificial Sequence
  PUMA forward primer sequence
tgcactgatggagatacggactt

SEQ ID NO: 54 - DNA, 19 bp, Artificial Sequence
  PUMA reverse primer sequence
cccctcggtcaccatgagt

SEQ ID NO: 55 - DNA, 16 bp, Artificial Sequence
  p21 forward primer sequence
gcgggaccgggacatc

SEQ ID NO: 56 - DNA, 26 bp, Artificial Sequence
  p21 reverse primer sequence
cgcttggagtgatagaaatctgttag

SEQ ID NO: 57 - DNA, 18 bp, Artificial Sequence
  p21 forward primer sequence
gattgcgatgcgctcatg

SEQ ID NO: 58 - DNA, 20 bp, Artificial Sequence
  p21 reverse primer sequence
cgtctcagtggcgaagtcaa

SEQ ID NO: 59 - DNA, 18 bp, Artificial Sequence
  p21 forward primer sequence
accagccacaggcaccat

SEQ ID NO: 60 - DNA, 21 bp, Artificial Sequence
  p21 reverse primer sequence
cggcatactttgctcctgtgt

(19) United States
(12) Patent Application Publication
(10) Pub. No.: US 2009/0298071 A1
(43) Pub. Date: Dec. 3, 2009

(54) METHOD FOR TESTING DRUG SENSITIVITY IN SOLID TUMORS BY QUANTIFYING MRNA EXPRESSION IN THINLY-SLICED TUMOR TISSUE

(76) Inventors: Masato Mitsuhashi, Irvine, CA (US); Kazuhiko Obara, Hitachi City (JP)

Correspondence Address: KNOBBE MARTENS OLSON & BEAR LLP, 2040 MAIN STREET, FOURTEENTH FLOOR, IRVINE, CA, 92614, US

(21) Appl. No.: 12/300,183

(22) PCT Filed: May 8, 2007
(86) PCT No.: PCT/US07/11121

§ 371 Date: Nov. 10, 2008

Related U.S. Application Data
(60) Provisional application No. 60/798,674, filed on May 8, 2006.

(51) Int. Cl. C12Q 1/68

(52) U.S. Cl. 435/6

(57) ABSTRACT

A method is disclosed for assaying the sensitivity of neoplastic tissue to therapeutic agents, and in particular, for the quantification of pro-apoptotic marker mRNA expression in cells obtained from thinly-sliced living tumor tissue in such methods. The method may comprise ascertaining a particular apoptosis marker mRNA for an individual tumor or tumor type as well as exposure of thin-sliced live cancer tissues from the individual tumor to candidate chemotherapeutic drug regimes in vitro, followed by an assessment of the level of the marker mRNA in the tissue.

### FIELD

The present disclosure relates to methods for assaying the sensitivity of neoplastic tissue to therapeutic agents, and in particular, relates to the quantification of pro-apoptotic marker mRNA expression in cells obtained from thinly-sliced living tumor tissue in such methods.

### DESCRIPTION OF THE RELATED ART

Patient-oriented “tailored”, “personalized”, or “individualized” medicine is a new concept that has come on the horizon recently, to identify suitable individualized treatment for each patient (see Jain K K, Curr Opin Mol Ther 4, 548 (2002), incorporated here by reference). It is particularly important for cancer patients, because chemotherapeutic agents are known to induce severe side effects. Current drug choice, however, relies on statistically significant results obtained by double blind clinical trials using large populations of patients. When a diagnosis is made, patients undergo statistically-proven standard treatments without considering individual variations in drug sensitivity. Although various attempts have been made to predict such drug responses, no universal methodology is available at this time, due mainly to 2 major reasons: 1) the characteristics of cancer cells may not be uniform once cancer cells are isolated in vitro from surgically removed tissues or biopsy specimens, and 2) the culture conditions do not generally mimic the normal physiological surroundings. Although the analysis of single nucleotide polymorphisms (SNPs) in genomic DNA provides some clue to identify sensitive or non-sensitive patients to particular drugs (see A. Di Paolo, R. Danesi, M. Del Tacca, Pharmacol. Res. 49, 331 (2004), incorporated here by reference), such information is not universally applicable to all drugs. Furthermore, even where a SNP that affects the response to a particular drug has been identified, it is still not known whether a second or third yet-to-be identified SNP compensates for, counteracts, or aggravates the change in response to the drug.

### SUMMARY

In an embodiment, a method of determining whether a therapeutic agent is likely to be effective against a solid tumor is disclosed, the method comprising: obtaining, from the solid tumor, first and second samples comprising substantially homogenous slices having a thickness of 20-500 micrometers; incubating the first sample in vitro with the agent; incubating the second sample in vitro with a control stimulus; after incubation, measuring the amount of an mRNA associated with apoptosis of the tumor cells in the first and second samples; and comparing the amounts of mRNA in the first and second samples, wherein the therapy is likely to be effective if the amounts are different by more than about 50%. In a further aspect, the substantially homogenous slices have a thickness within a range of approximately 50-200 micrometers. In a further aspect, each of the first and second samples comprise at least three homogeneous slices. In a further aspect, the control stimulus is selected from the group consisting of PBS and DMSO. In a further aspect, the first and second samples are incubated in a CO₂ incubator. In a further aspect, the first and second samples are incubated between three and five hours. In a further aspect, the first and second samples are incubated for approximately four hours. In a further aspect, the therapy is likely to be effective if the amount of the mRNA in the first sample is greater than the amount of the mRNA in the second sample. In a further aspect, comparing the amounts of mRNA in the first and second samples comprises determining a ratio of the amount of the mRNA in the first sample to the amount of mRNA in the second sample, and the therapy is likely to be effective if the ratio is 1.5 or greater. In a further aspect, the therapy is likely to be effective if the ratio is 2.0 or greater. In a further aspect, the therapeutic agent comprises a drug selected from the group consisting of daunorubicin, doxorubicin, cytarabine, cisplatin, etoposide, and mitoxantron.

In a further aspect, the mRNA associated with apoptosis of the tumor cells is identified by a method comprising: obtaining third and fourth samples from the solid tumor; exposing the third sample to a proapoptotic stimulus in vitro; incubating the third and fourth samples for a period of time sufficient to produce apoptotic changes in the cell samples; measuring the amount of at least one mRNA selected from the group consisting of p21, GADD, Apaf-1, SUMO, Bfl-1, BCL-W, BCL-2, PUMA, NOXA, Hrk, Bim, BINP3, Bik, Bid, Bad, Bcl-XS, Bok, Bak, Bax, LRP, and MRP in the third and fourth samples; and determining a ratio of the amount of the mRNA in the third sample to the amount of mRNA in the fourth sample, wherein an mRNA exhibiting a ratio of 1.5 or greater is identified as the apoptosis marker mRNA. In a further aspect, the third and fourth samples comprise substantially homogenous slices of the solid tumor. In a further aspect, the proapoptotic stimulus is radiation. In a further aspect, the period of time sufficient to produce apoptotic changes is between two and four hours. In a further aspect, the period of time sufficient to produce apoptotic changes is approximately three hours. In a further aspect, an mRNA exhibiting a ratio of 2.0 or greater is identified as the apoptosis marker mRNA.

In a further aspect, the first and second samples are obtained by a method comprising the steps of: removing a substantially homogeneous portion of a lesion from the solid tumor; embedding the portion in a material that has approximately the same hardness as the portion and does not reduce the viability of cells within the portion; bringing the temperature of the embedded portion to approximately 4° C.; and slicing the embedded portion. In a further aspect, the homogeneous portion is approximately cubical. In a further aspect, the cubical homogenous portion measures approximately 1 mm by 1 mm by 1 mm. In a further aspect, the material comprises nutrients and oxygen accessible to the cells. In a further aspect, the material is a gel. In a further aspect, the gel is produced from a liquid by application of a stimulus. In a further aspect, the stimulus is selected from the group consisting of a chemical agent, ultraviolet light, and electricity. In a further aspect, the embedding step comprises: immersing the substantially homogenous portion in a liquid; and converting the liquid to a gel by application of a stimulus.

### DETAILED DESCRIPTION OF THE PREFERRED EMBODIMENT

In order to overcome problems associated with changes in cancer cells placed in an in vitro environment, the present method exposes thin-sliced live cancer tissues to candidate chemotherapeutic drug regimes in vitro. Tissue sectioners, which are capable of preparing thin slices from live animal brain specimens, have been available in the neuroscience field for decades (see Mayahara H, Fujimoto K, Noda T, Tamura I, Ogawa K. Acta Histochem Cytochem. 14, 211 (1981), incorporated here by reference). However, they have never been used in preparing samples from human solid tumors for use in drug sensitivity assays. Live thin slices are ideal materials for such assays, because cancer specimens can be analyzed without disturbing cell-to-cell contacts. Although cells at both sides of the cut surfaces are killed, intact cell layers are maintained in the middle of the slices. Drugs penetrate into these intact cells, once such slices are suspended in appropriate culture media. Multiple identical slices can be prepared from a homogenous lesion of cancer mass, which may then be used for screening various drug regimens in vitro. It has up until now been unclear whether identical multiple thin slices (30-100 μm in thickness) can be cut from a fresh un-fixed cancer mass, since cancer tissue is much harder and more irregular than brain tissue. Moreover, it has up until now not been known how to maintain such removed slices under physiological conditions during in vitro treatment.

### Analysis of the Viability of Cells in Sectioned Tissue

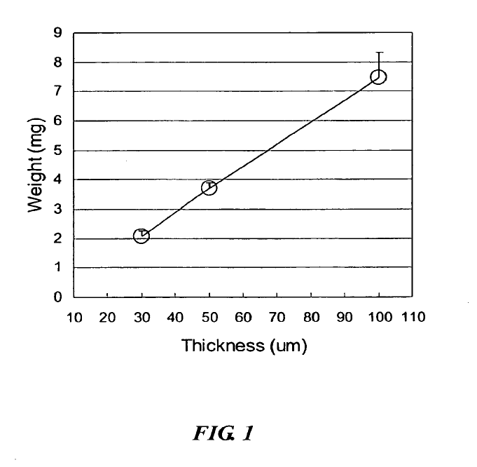

Pork tongue, the hardness of which is similar to solid tumor mass, was employed to assess the physical properties of sliced tissue. An approximately 10×5×5 (height) mm piece of this meat was cut by a razor blade, and placed onto a tissue sectioner (DSK-1000, Dosaka E M, Kyoto, Japan). The sample was placed in ice cold phosphate buffered saline (PBS, Invitrogen). Twelve slices of this sample were prepared, four of each thickness of 30, 50, and 100 μm, according to the instruction manual of the instrument. In order to evaluate the variation among quadruplicate samples, the dry weight of each slice was determined. In FIG. 1, the weight of the thin-sliced materials is shown. Quadruplicate data are shown as mean±standard deviation (s.d.). As shown in FIG. 1, the 3 data points corresponding to the 30, 50, and 100 μm slices were linear with a coefficient of variation (CV) of 5-12%.

In order to identify apoptotic changes, specimens are usually incubated at 37° C. overnight or longer. However, a long incubation period produces less physiological conditions and may induce some artifacts. Using previously identified early apoptotic markers, it was found that increases in pro-apoptotic mRNA expression could be identified after as little as 2-4 hours of incubation (see Mitsuhashi M, Tomozawa S, Endo K, Shinagawa A. Clin Chem. 52, 634 (2006); International Patent Application No. PCT/US2005/037925; and International Patent Application No. PCT/US2006/022427, all incorporated here by reference). This reason for this is that mRNA expression is an earlier event than protein synthesis and the resulting biological changes.

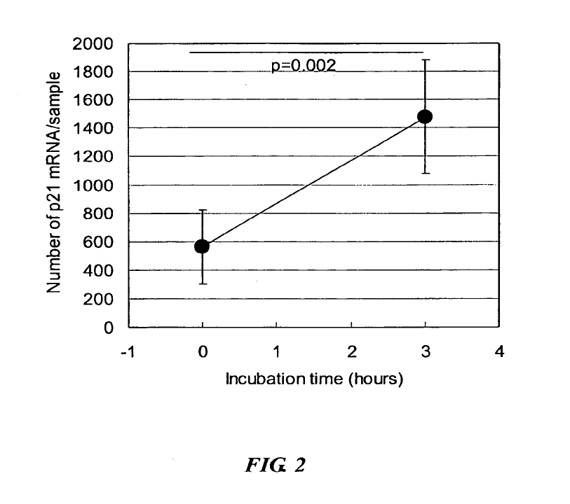

In an embodiment, 100 μm slices were prepared successfully from 2 cases of surgically removed stomach cancer specimens. The results of mRNA quantification in these samples are shown in FIGS. 2-4. p21 mRNA was quantified according to a method previously published by certain of the present inventors (see Mitsuhashi M, Tomozawa S, Endo K, Shinagawa A. Clin Chem. 52, 634 (2006), incorporated here by reference) from surgically removed stomach cancer specimens with or without 3 hours' incubation at 37° C. In brief, the slices were obtained using a tissue sectioner, and optionally incubated. Once the slices were ready for p21 mRNA quantification, lysis buffer was added thereto. The lysis buffer comprised, for example, 0.5% N-Lauroylsarcosine, 4×SSC, 10 mM Tris HCl, pH 7.4, 1 mM EDTA, 0.1% IGEPAL CA-630, and 1.791 M guanidine thiocyanate, supplemented with 1% 2-mercaptoethanol (Bio Rad, Hercules, Calif., USA), 0.5 mg/ml proteinase K (Pierce, Rockford, Ill., USA), 0.1 mg/ml salmon sperm DNA (5 Prime Eppendorf/Brinkmann, Westbury, N.Y., USA), 0.1 mg/ml E. coli tRNA (Sigma), a cocktail of 10 mM each of specific reverse primers, and standard RNA34 oligonucleotides. Then the tissue slices were dissolved by mixing the lysis buffer vigorously (pipetting up and down 10-20 times). The resultant lysis solution was transferred to a oligo(dT)-immobilized microplate (GenePlate, RNAture) by pipette. Following overnight storage at 4° C., the microplates were washed with 100 μl plain lysis buffer 3 times, followed by 150 μl of wash buffer (0.5 M NaCl, 10 mM Tris, pH 7.4, 1 mM EDTA) 3 times at 4° C. The cDNA was directly synthesized in each well by adding 30 μl of buffer containing 1×RT-buffer, 1.25 mM each of dNTP, 4 units rRNasin, and 80 units of MMLV reverse transcriptase (Promega) (without primers), and incubation at 37° C. for 2 hours. The specific primer-primed cDNA existed in solution, and oligo(dT)-primed cDNA stayed immobilized in the microplate. For TaqMan PCR, the resultant 4 μl of cDNA solution was directly transferred to 384-well PCR plates, to which 5 μl of TaqMan universal master mix (ABI) and 1 μl oligonucleotide cocktail (15 μM each of forward and reverse primer, and 3-6 μM TaqMan probe) were applied, and PCR was conducted in PRISM 7900HT (ABI), with one cycle of 95° C. for 10 min followed by 45 cycles of 95° C. for 30 sec, 55° C. for 30 sec, and 60° C. for 1 min. SYBR Green PCR may also be employed; for this, cDNA may be diluted 3-4 fold in water, and 4 μl cDNA solution directly transferred to 384-well PCR plates, to which 5 μl of a master mix (BioRad, Hercules, Calif.) and 1 μl of oligonucleotide cocktail (15 μM each of forward and reverse primer) is applied, and PCR then conducted in PRISM 7900HT (ABI), with one cycle of 95° C. for 10 min followed by 45 cycles of 95° C. for 30 sec and 60° C. for 1 min. Each gene was amplified in separate wells. The Ct was determined by analytical software (SDS, ABI).

In an embodiment, a quickly solidified inert gel may also be employed to embed the tissue mass before sectioning. For example, small disposable plastic cassettes containing a liquid gel that can be rapidly cured at 4° C. by the application of a chemical, ultraviolet light, or electricity may be employed. Once the tissue has been obtained, for example a cubical sample having dimensions of 1 mm on each side, it is placed in the cassette, and the liquid gel is then solidified. In an embodiment, the gel may be biologically inert so as not to damage the tissue cells. Also, in certain embodiments the hardness of the gel may be similar to that of the tissue. Cutting is then carried out, preferably at 4° C. to prevent cellular damage. In further embodiments, the gel provides nutrients and oxygen to the embedded tissue.

The tissue-embedded gel is fixed onto the cutting table, and placed under cold culture media. The cutting procedure is done in the media solution. Thus, once each tissue slice is made, it floats in the culture media. Each slice may be grasped using tweezers and placed into tissue culture plates (48- or 24-well plate). Drugs to be screened are added into the culture plates. After drug incubation, the solution is aspirated, and lysis buffer is added; the lysis buffer described above, for example, may be employed. The tissue slices are incubated in the lysis buffer at 37° C. for 10 min. Then the tissue slices are dissolved by mixing the lysis buffer vigorously (pipetting up and down 10-20 times). The resultant lysis solution is transferred to a GenePlate by pipette.

In further embodiments, a cutting machine may be employed that employs a flow of culture media, whereby each slice is collected in a separate well of a 96-well filterplate. Then, the filterplate is removed from the cutting machine, and is placed onto a holder, which temporarily blocks the hole of the filterplate at the bottom. Culture media and drugs are then added into each well, and incubation is conducted for 1-4 hours. Because the hole is blocked, the solution remains in each well. The filterplate materials are preferably inert enough not to damage tissues and not to absorb drugs. The filterplate is removed from the holder, and placed over a collection plate, and 150 μl 5 mM Tris, pH 7.4, is applied. Following centrifugation at 120×g for 1 min at 4° C., 50 μl of cell samples are applied to each well and immediately centrifuged at 120×g for 2 min at 4° C., followed by washing of each well with 300 μl PBS once with centrifugation at 2000×g for 5 min at 4° C. Then, 60 μl of the stock lysis buffer described above is applied to the filterplate, followed by incubation at 37° C. for 10 min. The filterplate is then placed over oligo(dT)-immobilized microplates (GenePlate, RNAture), and centrifuged at 2000×g for 5 min at 4° C. Following overnight storage at 4° C., the microplate is washed with 100 μl plain lysis buffer 3 times, followed by 150 μl of wash buffer (0.5 M NaCl, 10 mM Tris, pH 7.4, 1 mM EDTA) 3 times at 4° C. The cDNA is directly synthesized in each well by adding 30 μl of buffer containing 1×RT-buffer, 1.25 mM each of dNTP, 4 units rRNasin, and 80 units of MMLV reverse transcriptase (Promega) (without primers), and incubation at 37° C. for 2 hours. The specific primer-primed cDNA exists in solution, and oligo(dT)-primed cDNA stays immobilized in the microplate. For TaqMan PCR, the resultant 4 μl of cDNA solution is directly transferred to 384-well PCR plates, to which 5 μl of TaqMan universal master mix (ABI) and 1 μl oligonucleotide cocktail (15 μM each of forward and reverse primer, and 3-6 μM TaqMan probe) are applied, and PCR is conducted in PRISM 7900HT (ABI), with one cycle of 95° C. for 10 min followed by 45 cycles of 95° C. for 30 sec, 55° C. for 30 see, and 60° C. for 1 min. SYBR Green PCR may also be employed; for this, cDNA may be diluted 3-4 fold in water, and 4 μl cDNA solution directly transferred to 384-well PCR plates, to which 5 μl of a master mix (BioRad, Hercules, Calif.) and 1 μl of oligonucleotide cocktail (15 μM each of forward and reverse primer) is applied, and PCR then conducted in PRISM 7900HT (ABI), with one cycle of 95° C. for 10 min followed by 45 cycles of 95° C. for 30 sec and 60° C. for 1 min. Each gene was amplified in separate wells. The Ct is determined by analytical software (SDS, ABI).

The probe and primer sequences employed in the measurement of mRNAs are shown in Table 1 below.

TABLE I
PROBE AND PRIMER SEQUENCES

| | Accession | | |
|---|---|---|---|
| mRNA | No. | Forward primer | Reverse primer |
| RNA34 | | AGCCCCCTCA CTCCCAAA | GGGTGCTGTG CTTCTGTGAA C |
| | | (SEQ ID NO: 1) | (SEQ ID NO: 2) |
| p21 | HS431A14 | TTCTGCTGTC TCTCCTCAGA TTTCT | GGATTAGGGC TTCCTCTTGG A |
| | | (SEQ ID NO: 3) | (SEQ ID NQ: 4) |
| | | TaqMan probe: FAM-CCACTCCAAA | |
| | | CGCCGGCTGA TC-TAMRA | |
| | | (FAM-SEQ ID NO: 5-TAMRA) | |
| GADD153 | S40706 | AGAACCAGGA AACGGAAACA GA | TCTCCTTCAT GCGCTGCTTT |
| | | (SEQ ID NO: 6) | (SEQ ID NQ: 7) |
| Apaf-1 | AF013263 | TGCGCTGCTC TGCCTTCT | CCATGGGTAG CAGCTCCTTC T |
| | | (SEQ ID NO: 8) | (SEQ ID NO: 9) |
| SUMO-1 | BC006462 | GGGTCAGAGA ATTGCTGATA ATCAT | CCCCGTTTGT TCCTGATAAA CT |
| | | (SEQ ID NO: 10) | (SEQ ID NO: 11) |
| Bfl-1 | U27467 | CACAGGAGAA TGGATAAGGC AAA | CATCCAGCCA GATTTAGGTT CAA |
| | | (SEQ ID NO: 12) | (SEQ ID NO: 13) |
| BCL-W | NM_004050 | TCCGGCGCACCTTCTCT | CCCAAAGACAAAGAAGGCTACAA |
| | | (SEQ ID NO: 14) | (SEQ ID NO: 15) |
| Bcl-2 | BC027258 | CATGTGTGTG GAGAGCGTCA A | GCCGGTTCAG GTACTCAGTC A |
| | | (SEQ ID NO: 16) | (SEQ ID NQ: 17) |
| PUMA | AF354654 | GGGCCCAGAC TGTGAATCCT | ACGTGCTCTC TCTAAACCTA TGCA |
| | | (SEQ ID NO: 18) | (SEQ ID NO: 19) |
| | | TaqMan probe: FAM-CCCCGCCCCA TCAATCCCA | |
| | | (FAM-SEQ ID NO: 20) | |
| NOXA | BC032663 | CTCAGGAGGT GCACGTTTCA | TTCCAAGGGC ACCCATGA |
| | | (SEQ ID NO: 21) | (SEQ ID NO: 22) |
| HRK | HSU76376 | GGGAGCCCAG AGCTTGAAA | GCGCTGTCTT TACTCTCCAC TTC |
| | | (SEQ ID NO: 23) | (SEQ ID NO: 24) |
| BIM | BC033694 | TCCAGGACAG TTGGATATTG TCA | TAAGGAGCAG GCACAGAGAA AGA |
| | | (SEQ ID NO: 25) | (SEQ ID NO: 26) |
| BNIP3 | BC021989 | AAATATTCCC CCCAAGGAGT TC | CGCTCGTGTT CCTCATGCT |
| | | (SEQ ID NO: 27) | (SEQ ID NO: 28) |
| BIK | U34584 | TCCTATGGCT CTGCAATTGT CA | GGCAGGAGTG AATGGCTCTT C |
| | | (SEQ ID NO: 29) | (SEQ ID NO: 30) |
| BID | BC036364 | CATACACTTT TTCTCTTTCC ATGACATC | GGGCATCGCA GTAGCTTCTG |
| | | (SEQ ID NO: 31) | (SEQ ID NO: 32) |
| BAD | BCO01901 | CAGGCCTATG CAAAAAGAGG AT | CGCACCGGAA GGGAATCT |
| | | (SEQ ID NO: 33) | (SEQ ID NO: 34) |
| Bcl-Xs | NM_001191 | GGCAGGCGAC GAGTTTGA | GTTCCCATAG AGTTCCACAA AAGTATC |
| | | (SEQ ID NO: 35) | (SEQ ID NO: 36) |
| BOK | NM_032515 | TCACATGCTG GTTGCTTAAT CC | GCACAAGGAC CCCATCACA |
| | | (SEQ ID NO: 37) | (SEQ ID NO: 38) |
| BAK | NM_001188 | CACGGCAGAG AATGCCTATG A | CCCAATTGAT GCCACTCTCA |
| | | (SEQ ID NO: 39) | (SEQ ID NO: 40) |
| BAX | AY217036 | TTTCTGACGG CAACTTCAAC TG | GGTGCACAGG GCCTTGAG |
| | | (SEQ ID NO: 41) | (SEQ ID NO: 42) |
| LRP | | CAGCTGGCCATCGAGATCA | TCCAGTCTCTGAGCCTCATGC |
| | | (SEQ ID NO: 43) | (SEQ ID NO: 44) |
| MRP | | CAATGCTGTGATGGCGATG | GATCCGATTGTCTTTGCTCTTCA |
| | | (SEQ ID NO: 45) | (SEQ ID NO: 46) |

As shown in FIG. 2, p21 mRNA was quantified from small slices (10 mm×5 mm×100 μm) of stomach cancer. Quadruplicate data were shown as mean±s.d. p21 is a cyclin-dependent kinase inhibitor that inhibits the activity of cyclin-CDK2 and cyclin-CDK4 complexes and serves as a regulator of cell cycle progression at the G1 phase. In certain tumor tissues, such as mammary tumors, glioma, and ovarian carcinoma, it has also been shown to contribute to apoptosis.

Although the CV of p21 mRNA (39-46%) was larger than that of FIG. 1, such CV was acceptable to draw a statistically significant (p=0.02) conclusion of p21 induction during 3 hours' incubation. This confirms previous indications (see Mitsuhashi M, Tomozawa S, Endo K, Shinagawa A. Clin Chem. 52, 634 (2006)), that p21 mRNA increased during in vitro incubation without any stimulation, and indicates that samples removed from stomach tumors are alive and functional.

Other mRNAs associated with apoptosis in tumor cells were also measured. When cells are determined to undergo apoptosis, several pro-apoptotic proteins are known to be activated. These include the so-called Bcl-2/BAX family genes, which include, among others, BAX, BAK, BOKk, Bcl-XS, etc. Truncated forms of BAX, such as the so-called BH3-only members, are also known to be pro-apoptotic; this group consists of BID, BAD, BIK, BIM, NOXA, PUMA (p53 upregulated modulator of apoptosis), etc.

These mRNAs are all associated with apoptotic responses in one or more tumor types. GADD153 (growth arrest and DNA damage-inducible gene) has been shown to mediate apoptosis in myeloma, hepatoma, and colon cancer. The Apaf-1 protein interacts with cytochrome C released by the mitochondria and dATP to form apoptosome complexes, which can activate caspase 9 and lead to apoptosis. Apaf-1 silencing or downregulation has been implicated in melanoma and glioblastoma. SUMO-1 (small ubiquitin-related modifier) is structurally related to ubiquitin, but when proteins are modified by SUMO (sumoylated) they appear to be protected from ubiquitin-mediated degradation. Furthermore, SUMO controls pathways important for the surveillance of genome integrity, and regulates the PML/p53 tumour suppressor network, a key determinant in the cellular response to DNA damage. Bfl-1 is a member of the Bcl-2 family; it suppresses p53-mediated apoptosis and exhibits cell proliferation and transforming activities. Elevated levels of Bfl-1 have been found in stomach and colon cancer, as well as in breast cancer. BCL-W is another member of the Bcl-2 family and has antiapoptotic effects. It has been implicated in the protection of stomach cancer cells. The Bcl-2 gene has antiapoptotic effects. It forms complexes with caspase-9 and Apaf-1, which prevent these proteins from forming the apoptosome and initiating the protease cascade leading to apoptosis. It is implicated in B-cell malignancies and chronic lymphocytic leukemia. PUMA (p53-upregulated modulator of apoptosis, also known as BBC3) is transcriptionally induced to induce apoptosis via the mitochondrial apoptotic pathway. It is transcriptionally activated by p53, and is also upregulated after endoplasmic reticulum stress, independently of p53 status. It is implicated in melanoma, colorectal cancer, head and neck cancer, and pancreatic cancer, among others. Although data are not presented here, NOXA mRNA may also be a useful marker gene: NOXA (also known as PMAIP1 or ARP) has been found to be highly expressed in adult T-cell leukemia and has a proapoptotic function in response to cellular damage, participating in the activation of caspase-9 and ensuing apoptosis. Hrk is an activator of apoptosis that interacts with Bcl-2. It has been implicated in astrocytic tumours and central nervous system lymphomas. Bim shares the short BH3 motif with most Bcl-2 homologues; it provokes apoptosis. It has been implicated in the development of mantle cell lymphoma. BINP3 is a proapoptotic Bcl-2 family protein that has been linked to malignant glioma. The Bik (Bcl-2-interacting killer) protein is another proapoptotic Bcl-2 family member; it has been implicated in B-cell lymphomas and breast cancer, and expression has also been observed in epithelial and lung cells. Bid (BH3 interacting domain death agonist) is a proapoptotic protein that is implicated in osteosarcoma and gastric cancers. Bad is another Bcl-2 proapoptotic family member that has been linked to B-cell lymphomas and colon cancer. Bcl-XS is another proapoptotic member of this family that has been linked to hepatocellular carcinoma, breast cancer, and ovarian cancer. Bok (Bcl-2 related ovarian killer) is another Bcl-2 proapoptotic family member that has been linked to ovarian cancer. Bak, another Bcl-2 homolog, is a strong promoter of apoptosis, and has been implicated in gastric and colorectal cancers. Bax (Bcl-2 associated X protein) is a proapoptotic protein that is implicated in numerous cancers, including acute and chronic lymphocytic leukemias, gastric and colorectal cancer, breast cancer, and pancreatic cancer. Expression of LRP (lung resistance protein) has been found in human fibrosarcomas, hepatocellular carcinoma, and acute myeloid leukemia. The MRP gene has been shown to be expressed in hepatocellular carcinoma and colorectal carcinoma.

It has not yet been clearly elucidated which mRNA is expressed at the transcriptional level in each type of cancer specimen during the development of drug-induced apoptosis. Specific mRNAs may be more dominantly expressed depending on the type of cells, the type of drug, or the dosage or degree of stimulus. Individual variation is also possible. Thus, in an embodiment, these apoptosis-related mRNAs may be screened for each cancer (lung, liver, breast, etc.) with several different drugs. Once specific mRNA markers are identified for an individual tumor or for a particular cancer type, these marker mRNAs can be used in drug screening.

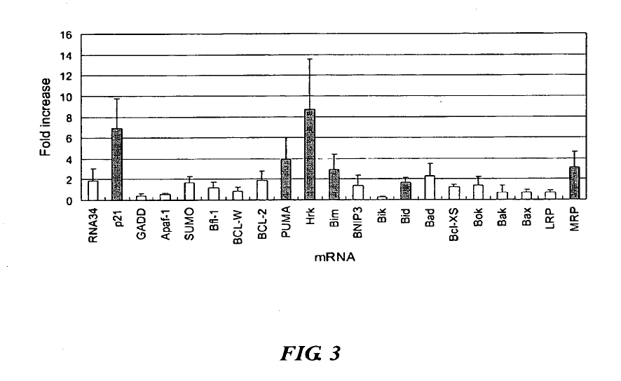

As shown in FIG. 3, not only p21, but also PUMA, Hrk, Bim, Bid, and MRP mRNAs were significantly (p<0.05) elevated during 3 hour's incubation. In FIG. 3, quadruplicate data are shown as mean±s.d. Shaded bars indicate p<0.05. The levels of control RNA (RNA34) spiked into the lysis buffer during mRNA preparation (see Mitsuhashi et al., Clin Chem. 52, 634 (2006), incorporated here by reference) did not show any significant changes, suggesting that the assay was performed appropriately.

Identification of Tumor-Specific mRNA Apoptosis Markers

In order to identify apoptotic changes, stomach cancer slices obtained as described above were stimulated with 15 Gy of radiation, and incubated at 37° C. for 3 hours. 15 Gy of radiation was chosen, in accordance with previous studies (see Mitsuhashi, et al., Clin Chem. 52, 634 (2006)). The levels of several apoptosis-associated mRNAs were then assessed using the methods described above.

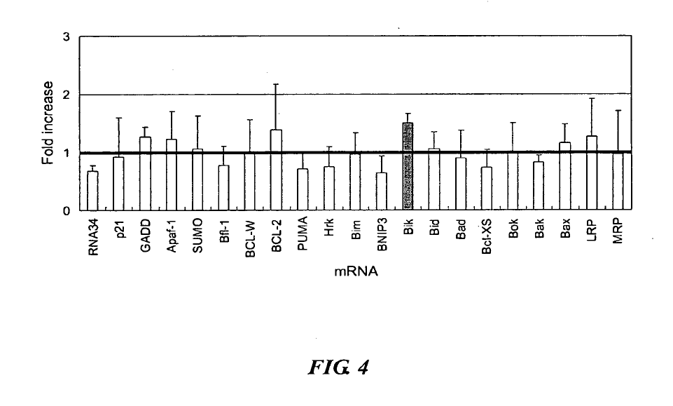

The results are shown in FIG. 4. In FIG. 4, quadruplicate data are shown as mean±s.d. Closed bars indicate p<0.05. As shown in FIG. 4, Bik, one of the pro-apoptotic BH3-only mRNAs, was significantly induced, although apoptotic marker mRNAs (p21 and PUMA) found in whole blood were not induced in this stomach cancer sample. In this case, Bik would be an appropriate marker for use in screening drug protocols for this tumor. Appropriate marker mRNAs may similarly be determined for each cancer. As data is developed through numerous cases for varying types of solid tumors, patterns of expression are expected to emerge which will allow clinicians to identify the most appropriate mRNA markers to employ in drug screening protocols based on histological, genetic, or other typing of the tumor. Once such a marker mRNA is identified for the individual tumor or selected based on the tumor type, live cancer slices may be used for mRNA analysis in vitro, which will make possible drug sensitivity assays for various cancer specimens.

Thus, in an embodiment of this method, slices taken from an individual tumor sample may be subjected to a proapoptotic stimulus such as radiation, and the stimulated slices and unstimulated slices may be incubated for a period of time sufficient to produce apoptotic changes in the cell samples. This may, for example, be for a period of approximately 3-4 hours. After this, the levels of various potential marker mRNAs linked to apoptosis in tumor tissues may be measured in the stimulated and unstimulated samples as described above. The marker mRNA may be one which is proapoptotic or antiapoptotic. In an embodiment, a proapoptotic mRNA exhibiting a ratio of the amount of mRNA in the stimulated samples to the amount in the unstimulated samples of 1.5 or more may be selected as a marker mRNA for use in drug screening. In a further embodiment, an mRNA exhibiting a ratio of 2.0 or greater may be selected. In a further embodiment, an antiapoptotic mRNA exhibiting a ratio of the amount of mRNA in the unstimulated samples to the amount in the stimulated samples of 1.5 or more may be selected as a marker mRNA for use in drug screening. In a further embodiment, an mRNA exhibiting a ratio of 2.0 or greater may be selected.

In a further embodiment, rather than obtaining slices of the tumor tissue for stimulation to identify a marker mRNA, the tumor tissue may be homogenized with collagenase or trypsin, and the isolated cell suspension is subjected to a stimulus, followed by measurement of the mRNAs.

### Drug Screening Protocol

Multiple thin slices (200 μm) were prepared from freshly isolated rat spleen, and these were incubated with various drugs for 4 hours in a CO₂ incubator. Spleen was employed as a proxy for tumor tissue because it is homogeneous, easily sectionable, and obtainable in large sizes. After incubation, β-actin (3 different primer sets), PUMA (1 primer set), and p21 (3 different primer sets) mRNAs were quantified by the method described above without using filterplates. The primer sequences are shown in Table 2 below.

TABLE 2
PROBE AND PRIMER SEQUENCES

| | | |
|---|---|---|
| mRNA | Forward primer | Reverse primer |

| | | |
|---|---|---|
| β-actin | TTCAACACCCCAGCCATGT | GTGGTACGACCAGAGGCATACA |
| (primer set 1) | (SEQ ID NO:47) | (SEQ ID NO: 48) |
| β-actin | TCTGTGTGGATTGGTGGCTCTA | CTGCTTGCTGATCCACATCTG |
| (primer set 2) | (SEQ ID NO: 49) | (SEQ ID NO: 50) |
| β-actin | CCCTGGCTCCTAGCACCAT | GAGCCACCAATCCACACAGA |
| (primer set 3) | (SEQ ID NO: 51) | (SEQ ID NO: 52) |
| PUMA | TGCACTGATGGAGATACGGACTT | CCCCTCGGTCACCATGAGT |
| | (SEQ ID NO: 53) | (SEQ ID NO: 54) |
| p21 | GCGGGACCGGGACATC | CGCTTGGAGTGATAGAAATCTGTTAG |
| (primer set 1) | (SEQ ID NO: 55) | (SEQ ID NO: 56) |
| p21 | GATTGCGATGCGCTCATG | CGTCTCAGTGGCGAAGTCAA |
| (primer set 2) | (SEQ ID NO: 57) | (SEQ ID NO: 58) |
| p21 | ACCAGCCACAGGCACCAT | CGGCATACTTTGCTCCTGTGT |
| (primer set 3) | (SEQ ID NO: 59) | (SEQ ID NO: 60) |

In brief, triplicate slices were exposed to 50 μM each of daunorubicin and doxorubicin (“DNR”), 1 mM of AraC and 50 μM cisplatin (“AraC”), and 500 μM etopiside and 50 μM mitoxantron (“VP16”), and respective controls (PBS for DNR and AraC, and DMSO for VP16) at 37° C. for 4 hours in a CO₂ incubator. Then β-actin (3 different primer sets), PUMA (1 primer set), and p21(3 different primer sets) mRNA were quantified without using filterplates. The resultant cDNA was diluted 4-fold in water, and 4 μl cDNA solution was directly transferred to 384-well PCR plates, to which 5 μl iTaq SYBR master mix (BioRad, Hercules, Calif.) and 1 μl oligonucleotide cocktail (15 μM each of forward and reverse primer) were applied, and PCR was conducted in PRISM 7900HT (ABI), with one cycle of 95° C. for 10 min followed by 45 cycles of 95° C. for 30 sec and 60° C. for 1 min. The 1×RT buffer was used as negative controls to confirm no primer dimer was generated under these PCR conditions. Moreover, the melting curve was analyzed in each case to confirm that the PCR signals were derived from the single PCR product. The Ct was determined by the analytical software (SDS, ABI). The ΔCt was calculated by substituting the Ct values of appropriate control samples, and fold increase was calculated by 2^{(-ΔCt)}, by assuming that the efficiency of each PCR cycle was 100%.

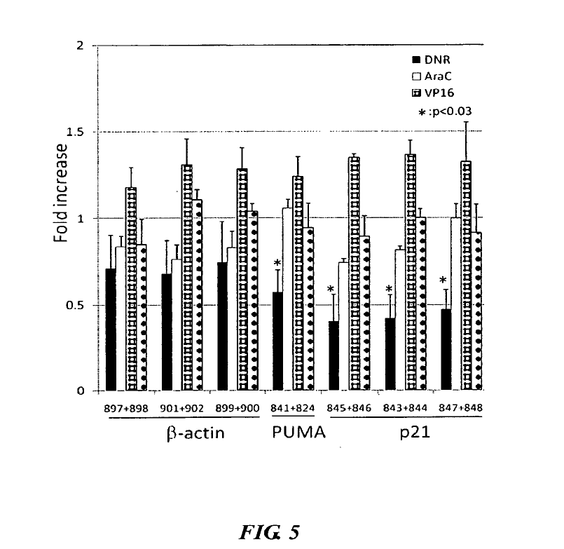

The results are shown in FIG. 5. In FIG. 5, the data are shown as the mean±s.d., and * indicates p<0.03. Although a slice-to-slice variation exists, a DNR-induced decrease in p21 and PUMA was identified with statistical significance, whereas the control housekeeping gene (β-actin) was unchanged. In contrast, the VP-16 and AraC regimens showed no statistically significant effect on mRNA expression in the tissue. This shows that the system is effective for assessing the sensitivity of the tissue to particular drug regimens.

Thus, in an embodiment, when tumor tissue exposed to a drug shows an altered level of expression of a marker mRNA linked to apoptosis in tumor tissue, this is indicative of the potential effectiveness of the therapy. In particular, when the level of the marker mRNA in tumor slices exposed to the drug and in tumor slices exposed to a control agent differ by more than about 50%, the therapy is likely to be effective. The marker mRNA may be proapoptotic or antiapoptotic. In a further embodiment, a ratio of the amount of proapoptotic mRNA in the slices exposed to the drug to the amount in the slices exposed to a control stimulus of 1.5 or more is indicative of an effective therapy. In a further embodiment, a ratio of 2.0 or greater is indicative of an effective therapy. In a further embodiment, a ratio of the amount of antiapoptotic mRNA in the slices exposed to the control stimulus to the amount in the slices exposed to a drug of 1.5 or more is indicative of an effective therapy. In a further embodiment, a ratio of 2.0 or greater is indicative of an effective therapy.

What is claimed is:

1. A method of determining whether a therapeutic agent is likely to be effective against a solid tumor, comprising:
obtaining, from the solid tumor, first and second samples comprising substantially homogenous slices having a thickness of 20-500 micrometers;
incubating the first sample in vitro with the agent;
incubating the second sample in vitro with a control stimulus;
after incubation, measuring the amount of an mRNA associated with apoptosis of the tumor cells in the first and second samples; and
comparing the amounts of mRNA in the first and second samples, wherein the therapy is likely to be effective if the amounts are different by more than about 50%.

2. The method of claim 1, wherein the substantially homogenous slices have a thickness within a range of approximately 50-200 micrometers.

3. The method of claim 1, wherein each of the first and second samples comprise at least three homogeneous slices.

4. The method of claim 1, wherein the mRNA associated with apoptosis of the tumor cells is identified by a method comprising:
obtaining third and fourth samples from the solid tumor;
exposing the third sample to a proapoptotic stimulus in vitro;
incubating the third and fourth samples for a period of time sufficient to produce apoptotic changes in the cell samples;
measuring the amount of at least one mRNA selected from the group consisting of p21, GADD, Apaf-1, SUMO, Bfl-1, BCL-W, BCL-2, PUMA, NOXA, Hrk, Bim, BINP3, Bik, Bid, Bad, Bcl-XS, Bok, Bak, Bax, LRP, and MRP in the third and fourth samples; and
determining a ratio of the amount of the mRNA in the third sample to the amount of mRNA in the fourth sample, wherein an mRNA exhibiting a ratio of 1.5 or greater is identified as the apoptosis marker mRNA.

5. The method of claim 4, wherein said third and fourth samples comprise substantially homogenous slices of the solid tumor.

6. The method of claim 1, wherein said first and second samples are obtained by a method comprising the steps of:
removing a substantially homogeneous portion of a lesion from the solid tumor;
embedding the portion in a material that has approximately the same hardness as the portion and does not reduce the viability of cells within the portion;
bringing the temperature of the embedded portion to approximately 4° C.; and
slicing the embedded portion.

7. The method of claim 6, wherein the homogeneous portion is approximately cubical.

8. The method of claim 7, wherein the cubical homogenous portion measures approximately 1 mm by 1 mm by 1 mm.

9. The method of claim 6, wherein the material comprises nutrients and oxygen accessible to the cells.

10. The method of claim 6, wherein the material is a gel.

11. The method of claim 10, wherein the gel is produced from a liquid by application of a stimulus.

12. The method of claim 11, wherein the stimulus is selected from the group consisting of a chemical agent, ultraviolet light, and electricity.

13. The method of claim 6, wherein the embedding step comprises:
immersing the substantially homogenous portion in a liquid; and
converting the liquid to a gel by application of a stimulus.

14. The method of claim 1, wherein the control stimulus is selected from the group consisting of PBS and DMSO.

15. The method of claim 1, wherein the first and second samples are incubated in a CO₂ incubator.

16. The method of claim 1, wherein the first and second samples are incubated between three and five hours.

17. The method of claim 16, wherein the first and second samples are incubated for approximately four hours.

18. The method of claim 1, wherein the therapy is likely to be effective if the amount of the mRNA in the first sample is greater than the amount of the mRNA in the second sample.

19. The method of claim 1, wherein comparing the amounts of mRNA in the first and second samples comprises determining a ratio of the amount of the mRNA in the first sample to the amount of mRNA in the second sample, and the therapy is likely to be effective if the ratio is 1.5 or greater.

20. The method of claim 19, wherein the therapy is likely to be effective if the ratio is 2.0 or greater.

21. The method of claim 4, wherein the proapoptotic stimulus is radiation.

22. The method of claim 4, wherein the period of time sufficient to produce apoptotic changes is between two and four hours.

23. The method of claim 4, wherein the period of time sufficient to produce apoptotic changes is approximately three hours.

24. The method of claim 4, wherein an mRNA exhibiting a ratio of 2.0 or greater is identified as the apoptosis marker mRNA.

25. The method of claim 1, wherein the therapeutic agent comprises a drug selected from the group consisting of daunorubicin, doxorubicin, cytarabine, cisplatin, etoposide, and mitoxantron.
